(19) United States
(12) Patent Application Publication
(10) Pub. No.: US 2005/0112177 A1
(43) Pub. Date: May 26, 2005

(54) Nutritional supplement composition and method

(76) Inventors: Minter H. Dopson, Warrenville, SC (US); Joseph H. Davis, West Columbia, SC (US); Michelle D. Dopson, Warrenville, SC (US); Robin P. Gunwall, Lexington, SC (US)

Correspondence Address: Nelson Mullins Riley & Scarborough, LLP, Post Office Box 11070, Columbia, SC, 29211-1070, US

(21) Appl. No.: 10/911,190

(22) Filed: Aug. 4, 2004

Related U.S. Application Data
(63) Continuation-in-part of application No. 10/324,637, filed on Dec. 20, 2002.

(51) Int. Cl. A23L 1/30; A61K 47/00

(52) U.S. Cl. 424/439

(57) ABSTRACT

The present invention provides a gelled composition containing a mixture of whey protein and other beneficial constituents, and a method for making the composition in a compact and easy-to-use form. The composition is made by processing a mixture of whey protein and other selected ingredients at an appropriate temperature and pH. The gel composition contains a surprisingly large fraction of whey proteins (and other beneficial constituents) in a readily bioavailable form. The composition can be packaged into conveniently-sized containers that are as easy to store or transport as they are easy to use.

### CROSS-REFERENCE TO RELATED APPLICATIONS

This application is a continuation-in-part of U.S. patent application Ser. No. 10/324,637, filed Dec. 20, 2002, and claims the priority benefit of that application, which is incorporated by reference herein in its entirety and i0s related to a commonly assigned and copending continuation-in-part application having the title “Nutritional Supplement Composition and Method” having Attorney Docket No. 24110/09000, which was filed on the same date as the present application.

### BACKGROUND OF THE INVENTION

(1) Field of the Invention

The present invention relates to a nutritional supplement composition. In particular, the present invention relates to a nutritional supplement composition containing whey protein, amino acids and other beneficial ingredients in a bioavailable form, and to a method for making the composition. The present invention also relates to food products that contain the composition, and to methods for using the composition.

(2) Description of the Related Art

Throughout recorded history, good nutrition has been recognized as one of the most important factors in maintaining good health. Protein in particular is recognized as an important nutrient, whether obtained from meat, dairy products, or other sources. Dairy products are widely used as part of a healthy diet: Indeed, the consumption of fermented whole milk (yogurt) may be associated with unusually long life expectancy in the parts of the former Soviet Union, particularly the Caucasus region.

Whey, also known as Serum lactis, is one of the by-products of the dairy industry. It is produced by acidifying milk at a pH near the isoelectric point of the casein, then separating the curd to recover the whey liquid. Alternative methods include flocculating or coagulating the milk caseins by adding rennet to whole milk, then separating out the whey liquid. So-called “sweet whey’ is produced by flocculation at a pH that is approximately that of milk, but is above about 5.8-6.0. Typically, sweet wheys are obtained from cooked or uncooked pressed curds such as those used in making Emmental, Gruyere, and Cheddar-type cheese. Acid wheys result from the processing of fresh curds and “mixed” wheys result from the processing of soft curds and marbled curds (i.e., curds used to make blue cheese).

The composition of whey varies depending on the composition of the starting milk and the selection of process. In general, whey contains fats, minerals, lactic acid, coagulated enzymes, and nitrogenous compounds, but lacks most of the fat, fat-soluble vitamins, caseins, and casein-bound calcium and phosphate found in whole milk or skim milk. For example, approximately 80% of the total protein content of cows milk, but only about 20% of the protein content of whey, consists of caseins. The protein fraction of whey includes a variety of soluble milk proteins, β-lactoglobulins, α-lactoalbumins, serum albumins, immunoglobulins, and amino acids.

Whey has traditionally been used as a fertilizer and as a constituent of animal feed. It is also a widely-used and well-documented folk remedy. Jaundice, skin infections, genito-urinary tract infections, venereal diseases, epilepsy, and fever have been treated with whey. More recently, it has been determined that regular consumption of whey results in an immuno-enhancing effect, and may contribute to detoxification of cancer-causing environmental agents. Whey is also an important dietary supplement used by athletes, bodybuilders, and others interested in achieving and maintaining optimum nutritional status, immune system status, and general health. Conditioning programs frequently include the consumption of large quantities of liquid whey several times daily. Thus, there is a large group of consumers who are interested in convenient dietary sources of whey.

A wide variety of nutritional supplements that contain whey, amino acids, and other beneficial ingredients are available to consumers. For purposes of this specification, the terms “nutritional supplement,” “food supplement,” and “dietary supplement” are used interchangeably. By way of example, Almada et al. (U.S. Pat. No. 5,726,146) disclose a non-steroidal anabolic dietary supplement and method for increasing lean body mass without increasing fat mass. The supplement includes creatine, taurine, ribonucleic acid, and a carbohydrate, and may include other ingredients such as alpha-ketoglutaric acid, beta-hydroxy-beta-methyl butyric acid, and salts thereof. The supplement may be consumed alone or combined with a nutrient base which includes proteins, carbohydrates, vitamins, minerals, and other amino acids.

Bounous, et al. (U.S. Pat. No. 5,451,412) and Gold et al. (U.S. Pat. No. 5,230,902) provide undenatured whey protein concentrates used as food supplements, and methods of making the concentrates. The concentrates contain proteins in an “essentially” undenatured state; their biological activity depends on the overall amino acid and small peptides pattern resulting from the protein components. Vitamins B1 and B2 may be added to further increase biological activity.

Henderson (PCT Application No. W000/37087) discloses a comprehensive 20 dietary supplement containing bioavailable minerals, vitamins, phytonutrients, herbs, antioxidants, and enzymes. The minerals are in the form of amino acid chelates.

Jarowski (U.S. Pat. No. 5,559,142) shows a universal dietary protein supplement which consists of a blend of four essential amino acids (L-Tryptophan, L-25 Methionine, L-Valine, L-Lysine Monohydrochloride). The relative proportions of at least the first three amino acids conform to the respective proportions found in human blood plasma (after fasting).

Maubois, et al. (U.S. Pat. No. 4,427,658) disclose a total enzymatic hydrolysate derived from whey proteins and a process for obtaining the hydrolysate. Their composition includes a peptidic hydrolysate with substantially no residual proteins, where at least 50% of the peptides contain 2-5 amino acids and 70%-90% of the nitrogen present in the peptides has less than 10 amino acids. The composition is used as a food supplement (in the form of pills), diet food, or intensive care food (administered via the enteral tract).

Meade (U.S. Pat. No. 5,631,031) describes a water-insoluble amino acid salt which can be used as a supplement for ruminant animals and humans. The supplement can be incorporated into flour or other food-compatible carriers.

O'Donnell, et al. (European Patent No. 1108 429 A2) provide an amino acid composition for use as an amino acid or protein supplement, aimed at the treatment and/or management of diseases of amino acid metabolism such as phenylketonuria. The composition includes a selection of amino acids, other standard nutrients (minerals, trace elements, vitamins, flavorings, etc), and is combined with a thickening agent such as pre-gelatinized starch, modified starch, gum, or cellulose.

Regnault, et al. (European Patent No. 1 208 749 AI and PCT Application 20 No. WO99/5517) describe a protein supplement, methods for preparing and using the supplement, and food compositions that contain the supplement. The supplement contains at least 25% (preferably 80%) protein by weight; the tryptophan transfer/neutral amino acid ratio is at least 0.06.

Shay, et al. (U.S. Pat. No. 4,439,525) disclose high methionine content yeasts of the strain Pichia pastoris. Their yeasts have good productivity in single cell protein production, and also have a higher amino acid content than other yeasts. The yeast cells are dried for use. The fermentor effluent can also be dried for use as a high-protein animal feed.

Whey is also used as a fertilizer. For example, Brinker et al. (U.S. Pat. No. 6,245,713) describe treatment compositions having enhanced biological effectiveness for application to the foliage of a plant for enhancing the efficiency of delivery to foliar-applied substances to their sites of action. The compositions include an herbicide, an alkylether surfactant, amine surfactants, and an amino group that is cationic or that can be protonated to become cationic. They are provided in solid or liquid concentrate form, and are mixed with water prior to use.

Kinnersley, et al. (U.S. Pat. Nos. 5,840,656 and 5,350,735) disclose water-soluble compositions, and methods for utilizing the compositions to enhance fertilizer uptake and promote plant growth. The compositions include water-soluble poly(organic acids), particularly poly(amino acids) such as poly(aspartic acid) and their copolymers, and can be applied by spraying.

Miyazawa, et al. (European Patent No. 1 208 749 AI and U.S. Application No. 20020095696) describe a tillering promoter that is applied to the leaf surfaces of plants to promote the production of side shoots by the plant. Their composition includes amino acids such as proline or inosine as the effective ingredients. U.S. Pat. No. 6,448,202 describes a withering-preventing agent that contains amino acids.

Despite the many different products that are available, no known product contains an effective amount of whey in a convenient, easy-to-use, easy-to-carry, and easy-to-store form. As noted above, bodybuilders and other athletes may consume several pints of liquid whey daily, preferably every 2-6 hours to maintain an even body load. Whey in liquid or solid form is readily available at health food stores, but is essentially impossible to find at the average restaurant or supermarket. Therefore, the average user who wishes to maintain a desired consumption schedule must carry his or her own supply to work or school. Liquid (i.e., bottled) whey is bulky and inconvenient to carry. Generally, liquids have small amounts of protein, meaning that the user must take in large volumes. Dehydrated, solid whey must be reconstituted in a blender, a common kitchen appliance which nevertheless is rarely found in the typical workplace.

Conventional methods for producing whey protein-based compositions must deal with whey's heat sensitivity issues and excessive foaming and/or denaturing of the whey protein. Typical processes for producing whey protein-based compositions involve pasteurization with traditional heat exchange devices such as plate heat exchangers. Pasteurization is the process of heating a consumable product at a specific temperature for a controlled period of time to destroy any remaining pathogenic organisms. Plate heat exchangers pass a thin film of product over a plate where a heating medium is being passed up or down on the other side of the plate. The thin film makes for rapid heat transfer and is a traditional method for heating nutritional based products during pasteurization, such as with the methods taught in U.S. Published Application No. 2003/0091613 to DeWille, et al.

Unfortunately, many whey varieties are quite heat sensitive at the presently accepted pasteurization temperatures of 165° F. to 170° F. for 1 minute. For example, the use of plate heat exchangers can become problematic in large process environments because whey protein often denatures and sticks to the heated plate surfaces at such high temperatures. For viscous gel-based products, such as the nutritional gel of the present invention, the problem of whey protein denaturing can become even more pronounced.

Yet another problem with traditional production methods for whey protein compositions, especially gel-based nutritional products, is the excessive amount of air entrapment into the gel. This creates not only undesirable bulk, but also the presence of oxygen potentially creates havoc in the microbiology and at the very least can shorten the shelf life of the final gel product.

Therefore, there remains a need for a nutritional composition that contains whey in a bioavailable form, that is cost-effective to manufacture, and that is easy and convenient to use. There also remains a need for an improved method for producing whey-protein based nutritional compositions and especially whey-protein based gel products.

### SUMMARY OF THE INVENTION

According to its major aspects and broadly stated, the present invention includes a nutritional supplement composition containing a mixture of whey protein and other beneficial constituents, and a method for making the composition in a nutritionally compact, easy-to-use form. The composition can be packaged into conveniently-sized containers that are as easy to store or transport as they are easy to use, and are shelf-stable without requiring refrigeration.

The present invention also provides a method for making the composition, which includes the step of processing a mixture of whey protein and other selected ingredients at an appropriate temperature and pH. By adjusting the pH of the mixture to within the approximate range of 2.5-5.0, and by processing at an appropriate temperature, it is possible to obtain a gel composition that contains a surprisingly large fraction of whey proteins (and other beneficial constituents) in readily bioavailable form.

Another feature of the present invention is the composition itself. As noted above, the composition contains a relatively large amount of whey (at least approximately 0.1 gram whey per mL water, preferably approximately 1 gram whey per mL water) as well as several optional ingredients such as amino acids (preferably low molecular weight amino acids), low-glycemic-index sugar, minerals (magnesium, calcium, zinc, manganese, chromium, selenium, molybdenum, potassium, boron, etc.), nutritionals (MSM, creatine, L-glutamine, taurine, malic acid, vitamins and vitamin precursors (vitamin A, carotene, cryptoxanthin, retinol, 3-dehydroretinol, vitamin C (ascorbic acid), vitamin E (tocopherol), etc.)), beneficial enzymes, and homeopathics. The composition may also include additional sources of protein including, but not limited to, egg protein and soy protein, as well as coloring and flavoring agents, fragrances, and other ingredients known and used in the art.

Still another feature of the present invention is the use of whey solids, which are an important constituent in many nutritional programs. Essentially any whey solids may be used with the invention, having molecular weights ranging between approximately 500 and 50,000, including mixed wheys resulting from the mixing of byproducts from the manufacture of different types of cheeses and whey solids prepared from raw or heat-treated milk by lactic or rennet precipitation followed by dehydration or evaporation.

Yet another feature of the present invention is a low-glycemic-index sugar, preferably Kiwi sugar or the like, which helps to optimize metabolizing of the composition and helps avoid the possibly deleterious effects of a sugar “high” which can result from over-consumption of sucrose. The use of low-glycemic-index sugar also makes the composition suitable for use by diabetics and others who must control their intake of dietary sugars. Other suitable sugars include, but are not limited to, stevia, saccharin, sucrolose, NUTRASWEET®, sucrose, and fructose.

Another feature of the present invention is the packaging. The end product composition is preferably packaged in containers made of moisture-barrier material, such as food-grade thermoplastic polymers, metal foil, and combinations thereof. The product is shelf-stable, does not require refrigeration or other special storage conditions, and can be opened and used “as is.” A single container may contain one or more servings of the composition. For example, containers with resealable bead and groove closures or other reclosable or resealable closures permit the end user to consume as much of the composition as he or she wishes, and save the rest for later use. Single-serving containers are convenient for packing with a “brown bag” lunch.

Still another feature of the present invention is the ability to use the composition in a variety of edible products including, but not necessarily limited to, nutraceuticals, dietary supplements, and pharmaceutical or veterinary compositions.

Yet another feature of the present invention is the use of the composition, and products containing the composition, as part of an overall health and fitness programs. The composition is broadly useful as a nutritional supplemental. Regular consumption of the composition (or products containing the composition) as part of a diet and exercise program helps the user build and maintain muscle mass and enhances overall health and well-being. The composition is also believed to help enhance the user's immune system when consumed on a regular basis.

Another feature of the present invention is an improved method for producing whey-protein-based nutritional compositions and especially whey-protein-based gel products.

Other features and advantages of the present invention will be apparent to those skilled in the art from a careful reading of the Detailed Description of Preferred Embodiments presented below and accompanied by the drawings.

### BRIEF DESCRIPTION OF THE DRAWINGS

FIG. 1 is a flow chart illustrating a method for making a composition according to a preferred embodiment of the present invention.

### DETAILED DESCRIPTION OF PREFERRED EMBODIMENTS

Reference now will be made in detail to the embodiments of the invention, one or more examples of which are set forth below. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used on another embodiment to yield a still further embodiment. Thus, it is intended that the present invention cover such modifications and variations as come within the scope of the appended claims and their equivalents. Other objects, features and aspects of the present invention are disclosed in or are obvious from the following detailed description. It is to be understood by one of ordinary skill in the art that the present discussion is a description of exemplary embodiments only, and is not intended as limiting the broader aspects of the present invention. In the following detailed description of the invention, the drawing is intended to be read together with the specification, and is to be considered a portion of the entire written description of this invention as required by 35 U.S.C. § 112.

As used herein, the term “comprise” and variations of the term, such as “comprising”, “comprises” and “comprised”, are not intended to exclude other additives, components, integers or steps.

Any reference to a numerical range in this application should be construed as an express disclosure of every number specifically contained within that range and of every subset of numbers contained within that range. Further, this range should be construed as providing support for a claim directed to any number, or subset of numbers in that range. For example, a disclosure of 1-10 should be construed as supporting a range of 2-8,3-7,5,6,1-9, 3.6-4.6,3,5-9.9, 1.1-9.9, etc.

The present invention is based on the discovery that a suitable mixture of whey protein and other beneficial constituents can be processed to form a gel composition. By processing the mixture at an appropriate temperature and pH, it is possible to obtain a gel composition that contains a surprisingly large fraction of whey proteins (and other beneficial constituents) in readily bioavailable form. The composition can be packaged into conveniently-sized containers that are as easy to store or transport as they are easy to use.

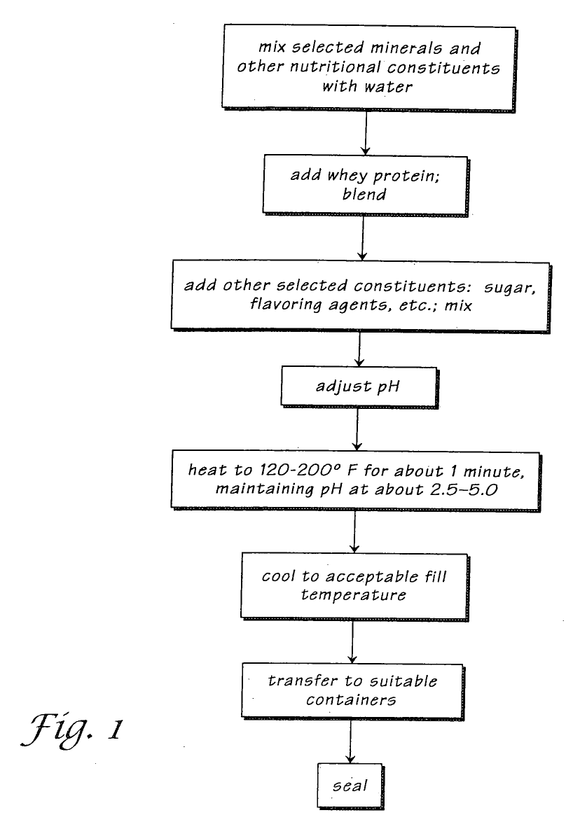

In accordance with a preferred embodiment of the invention, a nutritional composition containing whey is manufactured generally as shown in FIG. 1. The method for making the composition includes the following steps:

1. Adding, in some embodiments, a optional selected quantity of ingredients such as minerals, malic acid, and/or amino acids (preferably low molecular weight amino acids), and other nutritional constituents to water, and blending.

Suitable minerals include, but are not limited to, magnesium, calcium, zinc, manganese, chromium, selenium, molybdenum, potassium, boron, and mixtures thereof. Nutritional constituents that may be added to the composition include MSM, creatine, L-glutamine, taurine, malic acid, vitamins and vitamin precursors (vitamin A, carotene, cryptoxanthin, retinol, 3-dehydroretinol, vitamin C (ascorbic acid), vitamin F (tocopherol), etc.), beneficial enzymes, homeopathics, and flavoring agents. The composition may also include additional sources of protein, including but not limited to egg protein and soy protein.

2. Adding a selected quantity of whey protein, in solid form, to the water//nutritional blend and homogenizing.

Essentially any whey solids may be used with the invention, having molecular weights ranging between approximately 500 and 50,000. For example, mixed wheys resulting from the mixing of byproducts from the manufacture of different types of cheeses may be used with the invention. Alternatively, whey solids prepared from raw or heat-treated milk by lactic or rennet precipitation followed by dehydration or evaporation may also be useful. Preferably, the whey solids have been purified by ultrafiltration, microfiltration, diafiltration, ion exchange chromatography, or other suitable process prior to dehydration.

The ratio of whey solids to water may vary. Ratios between approximately 0.1-2 grams whey solids per mL of water are broadly suitable for the practice of the present invention. More preferably, the ratio is approximately 0.5-1.5 grams whey solids per mL of water, and most preferably, approximately 0.8-1.2 grams whey solids per mL of water. The optimum whey to water ratio depends on the other constituents of the composition and the nature of those constituents, as well as the desired end product.

3. Adding, in some embodiments, an optional quantity of sugar to the mixture.

Suitable sugars include, but are not limited to, low-glycemic-index sugars, Kiwi sugar, stevia, sucrolose, saccharin, NUTRASWEET®, fructose, glucose, and sucrose. Low-glycemic-index sugars are particularly useful, since these types of sugars do not produce the same elevated blood sugar levels after consumption as other types of sugars. While many factors influence an individual's blood sugar response to foods (including sugars), diabetics and those with impaired insulin response may benefit from consuming foods of relatively low glycemic index. Incorporation of low glycemic index foods in the diet is associated with reduced blood glucose, insulin, and lipid levels, as well as increased colonic fermentation, increased bacterial urea utilization, and increased production and absorption of short chain fatty acids in the colon.

4. If desired, adding additional constituents including, but not necessarily limited to, coloring agents, flavoring agents (vanilla, chocolate, strawberry, kiwi, orange, lemon, malt, etc.), fragrances, immune-system enhancers, antioxidants, fillers, stabilizers, antimicrobials, preservatives, pH-balancing agents, and combinations thereof.

Each ingredient of the composition is present in an amount that, as a percentage of the total weight of the composition, is effective either alone or synergistically with the other constituents to achieve the desired results. A composition according to the invention may contain approximately 5-50 wt. % whey (as added in Step 2 above), with the balance consisting of other constituents and water. Constituents such as minerals, vitamins, flavoring agents, and so forth may constitute 1-50 wt. % of the composition. However, amounts outside these ranges may also be useful.

5. In some embodiments, adjusting the pH of the mixture to approximately 2.5-5.0, and in other embodiments, to between about 3.5 to 4.0, and in still other embodiments to between about 3.6 and 3.8, by adding a malic acid or other suitable acid. The composition is naturally pH-balanced when formulated with selected ingredients as described below. However, pH-balancing agents may be added if needed.

6. Pasteurizing the pH-adjusted mixture.

Pasteurization can be carried out as a batch process, wherein the temperature of the mixture is raised to between approximately 120-200° F. for approximately between about one minute and one hour, and in some embodiments, for between about 10 minutes and 40 minutes, and in other embodiments, for between about 15 minutes and 30 minutes, and in still other embodiments, for about 30 minutes. In certain aspects of the invention, pasteurization is carried out at a temperature of between about 140° F. and about 160° F.; and in other aspects, at a temperature of between about 145° F. and about 150° F.

Alternatively, the mixture is processed by an in-line pasteurization system that elevates its temperature to the selected level for approximately one minute dwell time, and then cools the mixture to an acceptable fill temperature.

Irradiation, cold-sterilization, and/or other suitable processing techniques may be used instead of (or in addition to) pasteurization, provided that the composition contains a suitable amount of at least one food-grade constituent having preservative action. For example, grapefruit seed extract and other fruit-derived extracts may be useful.

7. After the mixture has cooled to an acceptable fill temperature, pumping into suitable containers such as pouches, gel-packs, and so forth, and sealing the filled containers.

The mixture may be packaged in any suitable container, preferably sterilizable containers made of moisture-barrier material. Suitable materials include food-grade thermoplastic polymers such as polystyrene, polyester, low density polyethylene, metal foil, and combinations thereof. For example, a suitable container might be a heat-sealable bag with walls that need be no more than 0.05-0.25 mm thick. Each container may contain one or more servings of the composition, as may be preferred. For example, a single-serving container might contain 1-2 ounces of the product, and measure no more than approximately 1-½by 4 inches (that is, about the size of typical granola bars, snack bars, and the like). Containers with resealable bead-and-groove closures or other recloseable or resealable closures permit the end user to consume as much of the composition as he or she wishes, and save the rest for later use.

In other embodiments, the present invention provides a novel method for the production of a whey protein-based nutritional product that has the form of a gel. A swept surface blending/heating vacuum vessel such as, for example, a swept surface blending/heating vacuum vessel, available from Lee Industries, Inc. can be used to mix and pasteurize the nutritional gel of the present invention at lower temperatures than traditional plate heat exchangers. Unlike plate heat exchangers, swept surface heat exchangers have blades that scrape the surface of the heat exchanger and bring new product continuously to the heating surface. This method helps to prevent prolonged contact between the surfaces of the heat exchanger (i.e., traditional plate exchanger surfaces) and the whey nutritional composition, thus helping to prevent denaturation of the whey proteins.

In some embodiments, the method involves first adding the whey protein and other ingredients into the swept surface vacuum vessel. The use of a swept surface vessel can allow the liquid portion of the nutritional composition to be introduced and then the solid ingredients added as a bulk and processed at atmospheric pressure. In other embodiments, the whey solids can also be introduced under a vacuum, which can create less foaming/denaturation.

In some embodiments, the swept surface vessel can mix and heat the ingredients while optionally holding them under a vacuum. The vessel can heat the mixture gradually while undergoing continuous stirring such as, for example from between about 10 to 100 revolutions per minute (rpm). For example, the temperature of the mixture can be slowly raised until reaching a pasteurization temperature of between about 120-200° F. for approximately between about one minute and one hour, and in some embodiments, for between about 10 minutes and 40 minutes, and in other embodiments, for between about 15 minutes and 30 minutes, and in still other embodiments, for about 30 minutes. In certain aspects of the invention, mixing and heating is carried out at a temperature of between about 140° F. and about 160° F.; and in other aspects, at a temperature of between about 145° F. and about 150° F.

The required time for mixing and adjusting the pH can also allow for gradual heating of the product without causing protein denaturation, while also providing the necessary pasteurization heat shock as required by GMP. The use of a swept surface heat exchanger allows, in some embodiments, the utilization of temperatures that are lower in comparison to traditional methods in order to prevent denaturing and sticking of the whey proteins.

In some embodiments, the nutritional composition can undergo a deaeration step in order to remove air entrapment and foaming in the final gel product. There are several styles of deaeration equipment, which would be suitable for use with the methods of the present invention. One advantageous type of deaeration unit can be a spinning disc separator with pick up assembly, which has a full vacuum for removal of even a small haze allowing for a clear gel that has a long shelf life. Another advantageous type of deaeration unit is a Versator™ vacuum deaerator, which is available from the Cornell Machine Co., Springfield, N.J.

In certain aspects, the method can encompass operating the swept surface vacuum vessel and the Versator™ deaerator simultaneously in a recirculation mode. However, the present method may, in certain aspects, still operate in a single pass through vacuum vessel deaerator. Operating in a recirculation mode can aid in the quick and efficient removal of air and may allow for an improved heat exchange (i.e., gels absorb heat more efficiently than foam), which can lead to a decreased process time. For example, the mixture can be mixed and heated in a swept surface vacuum vessel and then entered into a closed mix/heat recirculation loop with a vacuum deaeration device, such as the Versator™ vacuum deaerator. The mixture can then be passed through the Versator™ deaeration device one final time to ensure an air free final product and then moved on to the packaging process.

The several methods as described above results in a gel-type product that contains a surprisingly large amount of whey protein in an easy-to-use, easy-to-digest form. Instead of having to mix and drink large quantities of reconstituted liquid whey, a health-conscious consumer need only open a container of the composition and eat the contents. The product is shelf-stable, requiring no refrigeration or other special storage to maintain quality. The containers can be opened and the composition used ad libitum, without the need to heat, cool, reconstitute, or otherwise prepare the product for consumption.

The following examples describe various embodiments of the present invention. Other embodiments within the scope of the claims herein will be apparent to one skilled in the art from consideration of the specification or practice of the invention as disclosed herein. It is intended that the specification, together with the examples, be considered to be exemplary only, with the scope and spirit of the invention being indicated by the claims which follow the examples. In the examples, all percentages are given on a weight basis unless otherwise indicated.

### EXAMPLE 1

A mixture of malic acid and water was prepared. Whey protein solids, in an amount of 1 gram per 1 mL, were blended into the mixture. Sufficient malic acid was added in order to adjust the pH of the mixture to approximately 3.8. A sugar, such as Kiwi sugar was then added to the mixture being stirred in an amount of about 0.1 gram per 1 mL of mixture. The resulting mixture was heated to a temperature of about 145° F. for about 30 minutes, cooled, and transferred to flexible, heat-sealable containers, each container holding about 2 ounces of product.

The product had a gel-like consistency, and was determined to have an acceptable mouth “feel” and acceptable flavor as well as being more convenient than whey solids or reconstituted whey liquid.

A composition according to the present invention may be used as a nutritional supplement by athletes, bodybuilders, and others interested in achieving and maintaining optimum nutritional status. If desired, the composition can be incorporated into other edible products, including nutraceuticals, and pharmaceutical or veterinary compositions. It may also be administered parenterally or by mouth as a component of a therapeutic nutritional program for patients in intensive care, nursing home patients, and those with absorption problems. It may also be useful as a dietary supplement for children, including children who are reluctant to consume conventional vitamin supplements or who are “picky eaters”. The composition may also be useful as a general immune system toner or optimizer, since ingestion of whey is believed to enhance the immune system.

### EXAMPLE 2

Water at room temperature was placed in a Lee Industries, Inc. swept surface blending/heating vacuum vessel and stirred at a moderate speed. A mixture of whey protein solids (e.g., whey protein solids available from Proliant, Inc. in Ankeny, Iowa) at a concentration of 1 gram per 1 mL of mixture were added to the vessel. A sugar, such as Kiwi sugar was then added to the mixture being stirred in an amount of about 0.1 gram per 1 mL of mixture.

The vessel was allowed to heat the mixture gradually while undergoing continuous stirring. During the heating and stirring period, the pH of the mixture was adjusted to approximately 3.8 by addition of malic acid at 0.12 grams per 1 ml of mixture. The mixture was heated and stirred until reaching a temperature of between about 145° F. This temperature was held for approximately 30 minutes while the mixture entered into a closed mix/heat recirculation loop with a Versator™ vacuum deaeration device, which removed foam and air entrapment.

The mixture was then passed through the Versator™ deaeration device to promote an air-free final product. The resulting mixture was a nutritional composition in the form of a clear gel. The gel was then cooled and transferred to flexible, heat-sealable containers with each container holding about 2 ounces of gelled product.

In view of the above, it will be seen that the several advantages of the invention are achieved and other advantageous results obtained.

With respect to the above description of the invention, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

All references cited in this specification, including without limitation all papers, publications, patents, patent applications, presentations, texts, reports, manuscripts, brochures, books, internet postings, journal articles, periodicals, and the like, are hereby incorporated by reference into this specification in their entireties. The discussion of the references herein is intended merely to summarize the assertions made by their authors and no admission is made that any reference constitutes prior art. Applicants reserve the right to challenge the accuracy and pertinency of the cited references.

Although preferred embodiments of the invention have been described using specific terms, devices, and methods, such description is for illustrative purposes only. The words used are words of description rather than of limitation. It is to be understood that changes and variations may be made by those of ordinary skill in the art without departing from the spirit or the scope of the present invention, which is set forth in the following claims. In addition, it should be understood that aspects of the various embodiments may be interchanged both in whole or in part.

1. A nutritional composition comprising an aqueous solution in a gel form comprising:
(a) at least one whey protein;
(b) at least one sugar; and
(c) water.

2. The composition according to claim 1, wherein the whey protein comprises one or more whey protein solids.

3. The composition according to claim 2, wherein the whey protein solids are present in an amount of between about 0.8 and about 1.2 grams of whey protein solids per gram of water in the composition.

4. The composition according to claim 1, further comprising at least one or more of an ingredient chosen from minerals, vitamins, nutritional constituents, vitamin precursors, beneficial enzymes, amino acids, additional proteins, homeopathics, additional sugars, egg protein, soy protein, creatine, and mixtures thereof.

5. The composition according to claim 4, wherein the nutritional constituent comprises at least one constituent chosen from MSM, creatine, L-glutamine, taurine, beneficial enzymes, homeopathics, and mixtures thereof.

6. The composition according to claim 4, wherein the additional proteins comprise at least one protein chosen from egg protein, soy protein, and mixtures thereof.

7. The composition according to claim 1, further comprising the step of adding to said mixture at least one or more of any ingredient chosen from flavoring agents, coloring agents, fragrances, preservatives, and antimicrobials, and mixtures thereof.

8. The nutritional composition according to claim 1, wherein the sugar comprises at least one or more sugars chosen from kiwi sugar, stevia, sucrolose, saccharin, NUTRASWEET®, fructose, glucose, sucrose, natural sweeteners, artificial sweeteners, and mixtures thereof.

9. The method according to claim 4, wherein the sugar comprises at least one or more sugars chosen from low-glycemic-index sugars, kiwi sugars, stevia, and mixtures thereof.

10. The nutritional composition according to claim 1, wherein the sugar is a low-glycemic-index sugar.

11. The nutritional composition according to claim 1, wherein the sugar comprises a kiwi sugar.

12. The nutritional composition according to claim 1, wherein the sugar comprises stevia.

13. The nutritional composition according to claim 1, further comprising at least one ingredient chosen from minerals, vitamins, vitamin precursors, beneficial enzymes, homeopathics, egg protein, soy protein, and creatine.

14. The nutritional composition according to claim 1, further comprising an acid.

15. The nutritional composition as recited in claim 1, wherein the acid is malic acid.

16. The nutritional composition according to claim 1, wherein the composition has a pH of between about 2.5 and about 5.0.

17. The nutritional composition according to claim 1, wherein the composition has a pH of between about 3.5 and about 4.0.

18. The nutritional composition according to claim 1, wherein the composition has a pH of between about 3.5 and about 3.8.

19. The nutritional composition according to claim 1, wherein the composition has a pH of between about 3.6 and about 3.8.

20. The nutritional composition according to claim 1, wherein the composition contains between about 0.1 to about 2 grams of whey protein per mL of water.

21. The nutritional composition as recited in claim 1, further comprising at least one ingredient chosen from flavoring agents, coloring agents, fragrances, preservatives, antimicrobials, and mixtures thereof.

22. A nutritional composition comprising an aqueous solution in a gel form comprising:
(a) whey protein;
(b) at least one sugar;
(c) an acid; and
(d) water.

23. A nutritional composition comprising an aqueous solution in a gel form comprising:
(a) one or more whey protein solids;
(b) kiwi sugar;
(c) malic acid; and
(d) water, wherein the nutritional composition has a pH of between about 3.5 and about 4.0.
